(19) United States
(12) Patent Application Publication
(10) Pub. No.: US 2012/0141586 A1
(43) Pub. Date: Jun. 7, 2012

(54) THROMBIN RECEPTOR ANTAGONIST AND CLOPIDOGREL FIXED DOSE TABLET

(76) Inventors: Rubi Burlage, Westfield, NJ (US); Abdul S. Gafur, Clifton, NJ (US); Srinivas S. Duggirala, Raritan, NJ (US)

(21) Appl. No.: 13/376,633

(22) PCT Filed: Jun. 7, 2010
(86) PCT No.: PCT/US2010/037581

§ 371 Date: Feb. 20, 2012

Related U.S. Application Data
(60) Provisional application No. 61/185,068, filed on Jun. 8, 2009.

(51) Int. Cl. A61K 31/4365; A61P 9/00; A61P 9/06; A61P 9/12; A61P 9/04; A61K 9/24; A61P 9/10

(52) U.S. Cl. 424/465; 514/301; 424/464

(57) ABSTRACT

The present invention provides for a pharmaceutical formulation which comprises a) a compound of the formula (I): SCH 530348 or the bisulfate salt thereof; b) clopidogrel; and c) silicified microcrystalline cellulose.

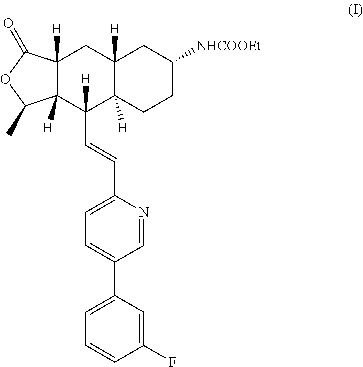

### RELATED APPLICATIONS

This application claims the benefit of U.S. provisional application U.S. Ser. No. 61/185,068, filed Jun. 8, 2009, herein incorporated by reference.

### FIELD OF THE INVENTION

The present invention relates to a pharmaceutical formulation, e.g., tablet such as a bilayer tablet, comprising the thrombin receptor antagonist SCH 530348 or the bisulfate salt thereof in combination with clopidogrel (i.e, the free base form of clopidogrel bisulfate).

### BACKGROUND OF THE INVENTION

Merck & Co. Inc. is developing a thrombin receptor antagonist (“TRA”) for use in a variety of cardiovascular applications, including treatment of acute coronary syndrome (“ACS”) and secondary prevention. The active pharmaceutical ingredient (“API”), is SCH 530348 (i.e, the free base form) and/or the bisulfate salt of SCH 530348 (“SCH 530348 bisulfate”). This compound has completed phase I and II clinical trials, and is currently in phase III trials.

Thrombin is known to have a variety of activities in different cell types and thrombin receptors are known to be present in such cell types as human platelets, vascular smooth muscle cells, endothelial cells and fibroblasts. It is therefore possible that thrombin receptor antagonists, also known as protease activated receptor (PAR) antagonists, will be useful in the treatment of thrombotic, inflammatory, atherosclerotic and fibroproliferative disorders, as well as other disorders in which thrombin and its receptor play a pathological role.

U.S. Pat. No. 7,304,078 discloses a genus of compounds, including SCH 530348 and SCH 530348 bisulfate (see Example 2). SCH 530348 or ethyl[(1R,3aR,4aR,6R,8aR,9S,9aS)-9-[(E)-2-[5-(3-fluorophenyl)-2-pyridinyl]ethenyl]dodecahydro-1-methyl-3-oxonaphtho[2,3-c]furan-6-yl]carbamate has the following structure:

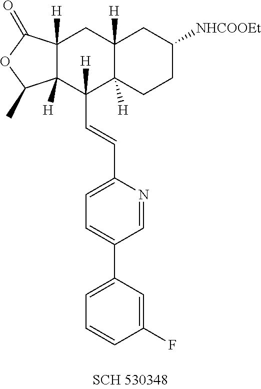

The bisulfate salt of SCH 530348 has the following structure:

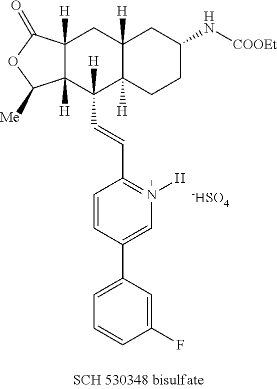

SCH 530348 and its bisulfate salt exhibit good thrombin receptor antagonist activity (potency) and selectivity. U.S. Publication No. 2004/0192753 (U.S. Ser. No. 10/705,282), herein incorporated by reference, discloses a variety of indications and combination formulations for thrombin receptor antagonists including SCH 530348 and its bisulfate salt. A preferred crystalline form of the bisulfate salt of SCH 530348 bisulfate is disclosed in U.S. Pat. No. 7,235,567. U.S. Publication Nos. 2008/0026050 (U.S. Ser. No. 11/771,571); 2008/0817821 (U.S. Ser. No. 11/771,520); and 2008/0152712 (U.S. Ser. No. 11/860,165) disclose capsule formulations, tablet formulations and lyophilized formulations (respectively) of SCH 530348 bisulfate, and methods of treating various conditions by administering same.

The use of a small subset of thrombin receptor antagonists to treat a variety of conditions and diseases is disclosed in U.S. Publication No. 2004/0192753. The prevention of complications associated with cardiopulmonary bypass surgery by administration of a thrombin receptor antagonist is taught in U.S. Publication No. 20070202140 (U.S. Ser. No. 11/613,450). Methods of preventing cardiac events after percutaneous intervention (“PCI,” e.g., angioplasty, stent introduction) are disclosed in U.S. Publication No 2008/0234236 (U.S. Ser. No. 12/051,504). Substituted thrombin receptor antagonists are disclosed in U.S. Pat. Nos. 6,063,847; 6,326,380; and 6,645,987 and U.S. Publication Nos. 2003/0203927; 2004/0216437A1; 2004/0152736; and 2003/0216437. All of the herein cited references are incorporated in their entirety.

Clopidogrel is a compound disclosed in U.S. Pat. No. 4,847,265 and taught to be therapeutically useful as an inhibitor of ADP-induced platelet aggregation. Tablets containing clopidogrel as clopidogrel bisulfate are sold in the United States and elsewhere under the tradename PLAVIX® by Bristol-Myers Squibb and Sanofi-Aventis. PLAVIX® is approved for reduction of artherothrombotic events such as recent myocardial infarction, recent stroke, established peripheral artery disease, and acute coronary syndrome with aspirin.

Clopidogrel, in its free base form, is an amorphous sticky, glue-like material that has technically challenging handling and processing properties.

Two active agents may be delivered as either co-administered monotherapy formulations, or as a single co-formulation and provide particularly beneficial therapeutic results. Co-formulations have the patient-compliance advantages of reducing the number of distinct doses and fixing the ratio of the two active agents being administered. The use of SCH 530348 or its bisulfate salt and PLAVIX® as a combination therapy has been evaluated and determined to be of a great benefit in treating cardiovascular diseases and disorders.

The difference between the physicochemical properties of SCH 530348 or its bisulfate salt and clopidogrel free base presents a substantial challenge in developing a SCH 530348 and/or SCH 530348 bisulfate-clopidogrel co-formulation. Physiochemical differences between the two active ingredients might result in the loss of the benefits of the overall combination therapy. A possible way to formulate two chemically incompatible active ingredients, among others, is to formulate the active ingredients as a bilayer tablet.

In light of these significant challenges in formulating two active agents with different physicochemical properties, an object of the present invention, among others that will be apparent from this description, is to provide physically and chemically stable formulation comprising SCH 530348 and/or its bisulfate salt and clopidogrel, formulated such as, for example as a bilayer tablet, that would provide these and other benefits, which will become apparent as the description progresses.

### SUMMARY OF THE INVENTION

The pharmaceutical formulations of the present invention addresses, inter alia, the aforementioned challenges. For example, one challenge is that chlopidogrel free base is very difficult to formulate because it is amorphous and as a sticky, glue-like property. Even when in the form of a premix, this material is not in a form that may easily formulate as, for example, a tablet.

Another significant challenge in developing a co-formulation containing SCH 530348 and/or its bisulfate salt and clopidogrel is the observation that clopidogrel undergoes significant degradation in the presence SCH 530348 bisulfate in a monolayer tablet formulation, i.e. where there is some reasonable level of exposure of the clopidogrel to SCH 530348 bisulfate. However, it was observed that clopidogrel is more stable when the SCH 530348 bisulfate and clopidogrel free base are compressed as two separate layers of a bilayer tablet, but the differences in the physicochemical properties of the clopidogrel premix and the SCH 530348 bisulfate make forming a stable tablet formulation a significant challenge. The selection of the excipient list, the amount of each excipient and the techniques used in handling the recipients along with each of the active ingredients are important to achieving the pharmaceutically desired structural tablet integrity, tablet size and overall tablet stability.

The clopidogrel free base is adsorbed onto pharmaceutically-acceptable excipients to form a clopidogrel free base “premix”. This clopidogrel premix may be obtained from Dr. Reddy's Laboratories LTD., and is disclosed in PCT Pub. No. WO 2006/044548 A2, which is herein incorporated by reference in its entirety.

The present invention provides for a pharmaceutical formulation which comprises:

a) a compound of the formula:

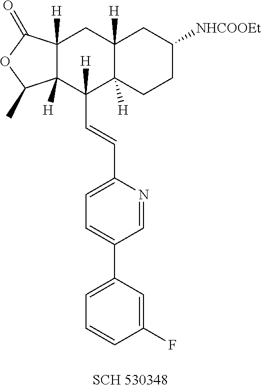

or the bisulfate salt thereof;

b) clopidogrel; and

c) silicified microcrystalline cellulose.

Examples of such formulations include tablets, such as bilayer tablets wherein the one layer comprises, for example, SCH 530348 and/or SCH 530348 bisulfate and the other layer comprises clopidogrel.

The present invention also provides for a bilayer pharmaceutical tablet prepared by dry granulation, which comprises: (a) a blend of SCH 530348 and/or SCH 530348 bisulfate, anhydrous lactose, silicified microcrystalline cellulose, croscarmellose sodium, hydroxypropyl cellulose and magnesium stearate; and (b) a blend of clopidogrel premix, anhydrous lactose, silicified microcrystalline cellulose, croscarmellose sodium and magnesium stearate as well as a method to prepare the same. The two blends exhibit sufficient mechanical and physical attributes for roller compaction, milling, further blending and bilayer tablet compression of blend (a) and blend (b). The bilayer tablet is coated with an aqueous film coating suspension; e.g. OPADRY® II film coating system and OPADRY® Fx™ film coating system, available from Colorcon. In certain embodiments, the formulations of the present invention may optionally further comprise one or more additional pharmaceutically acceptable excipients.

In some embodiments, the invention is directed to a pharmaceutical formulation which comprises SCH 530348; i.e., the free base.

In some embodiments, the invention is directed to a pharmaceutical formulation which comprises SCH 530348 bisulfate.

In some embodiments, the invention is directed to a pharmaceutical formulation which comprises SCH 539348 and SCH 530348 bisulfate.

In some embodiments, the invention is directed to a pharmaceutical tablet which comprises:

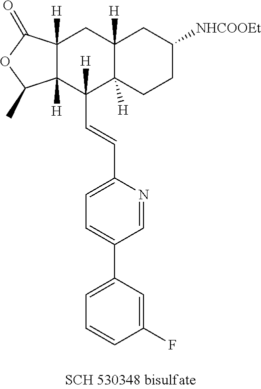

and clopidogrel and silicified microcrystalline cellulose.

In some embodiments, the invention is directed to a pharmaceutical tablet which is a bilayer tablet.

In some embodiments, the invention is directed to a pharmaceutical tablet comprising a first layer containing SCH 530348 bisulfate and a second layer containing clopidogrel.

In some embodiments, the bilayer tablet further comprises one or more other excipients.

In some embodiments, the other excipients of said first layer is selected from the group consisting of anhydrous lactose, silicified microcrystalline cellulose, sodium croscarmellose, hydroxypropyl cellulose and magnesium stearate; and the excipients of said second layer is selected from the group consisting of butylated hydroxyl anisole, mannitol, anhydrous lactose, micorocyrstalline cellulose, silicon dioxide, silicified microcrystalline cellulose, sodium croscarmellose, and magnesium stearate.

In some embodiments, the pharmaceutical formulation is coated.

In some embodiments, the invention is directed to a method of treating cardiovascular conditions comprising administering to a mammal in need of such treatment an effective amount of a pharmaceutical formulation comprising SCH 530348 and/or its bisulfate salt, clopidogrel and microcrystalline cellulose.

In some embodiments, the invention is directed to a method of treating a cardiovascular condition comprising administering to a mammal in need of such treatment an effective amount of the SCH 530348 bisulfate-clopidogrel bilayer tablet.

In some embodiments, the invention the cardiovascular condition is selected from the group consisting of acute coronary syndrome, peripheral arterial disease, thrombosis, atherosclerosis, restenosis, hypertension, angina pectoris, arrhythmia, heart failure, myocardial infarction, glomerulonephritis, thrombotic stroke, thromboembolic stroke, and cerebral ischemia.

In some embodiments, the cardiovascular condition is acute coronary syndrome.

In some embodiments, the cardiovascular condition is peripheral arterial disease.

In some embodiments, the invention is directed to preventing a condition associated with coronary arterial bypass graft surgery comprising administering to a subject of said surgery an effective amount of a pharmaceutical formulation comprising SCH 530348 and/or its bisulfate salt, clopidogrel and microcrystalline cellulose.

In some embodiments, the invention is directed to preventing a condition associated with coronary arterial bypass graft surgery comprising administering to a subject of said surgery an effective amount of a SCH 530348 bisulfate-clopidogrel bilayer tablet.

In some embodiments, the condition associated with coronary arterial bypass graft surgery is selected from the group consisting of: bleeding; thrombotic vascular events such as thrombosis, restenosis; vein graft failure; artery graft failure; atherosclerosis, angina pectoris; myocardial ischemia; acute coronary syndrome myocardial infarction; heart failure; arrhythmia; hypertension; transient ischemic attack; cerebral function impairment; thromboembolic stroke; cerebral ischemia; cerebral infarction; thrombophlebitis; deep vein thrombosis; and, peripheral vascular disease.

In some embodiments, the invention is directed to a method of preventing a major cardiac event in a patient who has undergone percutaneous coronary intervention and is in need of such prevention comprising administering an effective amount of a pharmaceutical formulation comprising SCH 530348 and/or its bisulfate salt, clopidogrel and microcrystalline cellulose.

In some embodiments, the invention is directed to a method of preventing a major cardiac event in a patient who has undergone percutaneous coronary intervention and is in need of such prevention comprising administering an effective amount of a SCH 530348 bisulfate-clopidogrel bilayer tablet to the patient.

In some embodiments, the major cardiac event is a myocardial infarction, urgent revascularization, or ischemia requiring hospitalization.

In some embodiments, the bilayer tablet provides the amount of SCH 530348 bisulfate in one layer of the bilayer tablet equivalent to about 2.5 mg and the amount of clopidogrel in the second layer of the bilayer tablet equivalent to about 75 mg.

In some embodiments, the compression aid is silicified microcrystalline cellulose.

In some embodiments, the compression aid is anhydrous lactose.

In some embodiments, the ratio of silicified microcrystalline cellulose to anhydrous lactose is 3:1.

In some embodiments, the binder is hydroxylpropyl cellulose EXF.

In some embodiments, the disintegrant is croscarmellose sodium.

In some embodiments, the lubricant is magnesium stearate.

In some embodiments, the sub-coat is OPADRY® II Orange.

In some embodiments, the top-coat is OPADRY® Fx™ Yellow.

In some embodiments, the coat is a one-step OPADRY® Fx™ coat.

### BRIEF DESCRIPTION OF THE DRAWINGS

FIG. 1: Process Flow Diagram for the Manufacture of SCH 530348 bisulfate-clopidogrel bilayer tablets (2.5 mg/75 mg).

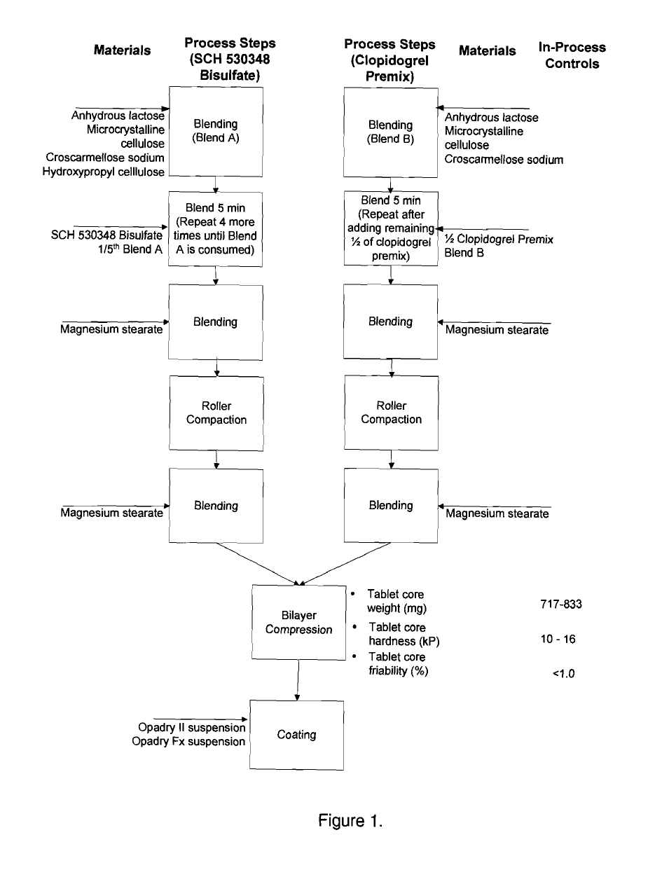

FIG. 2: Bar chart diagram showing clopidogrel degradation in the presence of SCH 530348 bisulfate, which is shown in comparison to SCH 530348 in its free base form.

### DETAILED DESCRIPTION OF THE INVENTION

### Definitions

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety. Any reference to SCH 417891 is clopidogrel premix. Any reference to SCH 900423 is the SCH 530348-clopidogrel bilayer (2.5 mg/75 mg) tablet.

As used above, and throughout this disclosure, the following terms, unless otherwise indicated, shall be understood to have the following meanings:

“Patient” includes both human and animals.

“Mammal” means humans and other mammalian animals.

“Granulation” refers to the process of agglomerating powder particles into larger granules that contain the active pharmaceutical ingredient.

“Dry granulation” refers to any process comprising the steps where there is no addition of a liquid to powdered starting materials, agitation, and drying to yield a solid dosage form. The resulting granulated drug product may be further processed into various final dosage forms, e.g., capsules, tablets, wafers, gels, lozenges, etc.

The raw material in a dry granulation process is typically the active pharmaceutical ingredient in a powder form, but could also be in a paste form. The powder or paste form can be generated by grinding, and the particle size distribution that results from the grinding step may influence the properties of the formulation. The active pharmaceutical ingredient may be mixed with other excipients, such as binders, disintegrants, fillers, or lubricants, into a powder blend.

After the granulation has been applied to the powder mix, and sufficient agitation has been applied to agglomerate the powder particles into granules, the granules are dried and milled. After milling, additional excipients may be added to form the final blend. Such additional excipients may include binders, disintegrants, fillers, and lubricants. The final blend is then compressed into the desired solid dosage form, e.g., tablets.

Pharmaceutical formulations of the present invention can be prepared, for example, using the following dry granulation process.

### Processing SCH 530348 Bisulfate Layer

Step 1. Blend ⅕ of the SCH 530348 bisulfate with anhydrous lactose, silicified microcrystalline cellulose, sodium croscarmellose and hydroxypropyl cellulose for 5 minutes and repeat 4 additional times, adding an additional ⅕ of the SCH 530348 bisulfate each time to form Blend A.

Step 2. Blend magnesium stearate into Blend A

Step 3. Roller Compact Blend from Step 2 and mill into granules.

Step 4. Blend remaining magnesium stearate into granules from step 3.

Processing Clopidogrel Layer by Dry Granulation

Step 1. Blend ½ of the procured clopidogrel premix (which contains amorphous clopidogrel in its free base form, butylated hydroxyl anisole (BHA), mannitol, lactose anhydrous, microcrystalline cellulose and silicon dioxide) with anhydrous lactose, silicified microcrystalline cellulose, and sodium croscarmellose for 5 minutes and repeat after adding remaining ½ of procured clopidogrel premix to form Blend B.

Step 2. Blend magnesium stearate into Blend B

Step 3. Roller Compact Blend from Step 2 and mill into granules.

Step 4. Blend in remaining magnesium stearate into granules from step 3.

Bilayer Compression

Compress the SCH 530348 bisulfate layer with clopidogrel layer to yield a bilayer tablet and coat with an OPADRY® II suspension and OPADRY® Fx™ suspension.

### EXAMPLES

(1): The formulations according to the present invention were prepared as described above and described in Tables 1-1 and 1-3.

TABLE 1-1
Composition of SCH 530348 Bisulfate-Clopidogrel
Bilayer Tablets; 2.5 mg/75 mg

| | | |
|---|---|---|
| | | Amount/Tablet |
| Ingredient | Function | (mg) |

Clopidogrel Layer

| | | | |
|---|---|---|---|
| 1. | Clopidogrel Premix | Active | 375* |
| 2. | Silicified microcrystalline | Compression aid | 129.5 |
| | cellulose | | |
| 3. | Lactose anhydrous | Compression aid | 43.2 |
| 4. | Croscarmellose sodium | Disintegrant | 23.0 |
| 5. | Magnesium stearate | Lubricant | 4.3 |
| | | Total layer weight | 575.0 |

SCH 530348 bisulfate Layer

| 6. | TRA-Bisulfate | Active | 2.5 |
|---|---|---|---|
| 7. | Silicified microcrystalline | Compression aid | 135.4 |
| | cellulose | | |
| 8. | Lactose anhydrous | Compression aid | 45.1 |
| 9. | Croscarmellose sodium | Disintegrant | 8.0 |
| 10. | Hydroxypropyl cellulose | Binder | 8.0 |
| 11. | Magnesium stearate | Lubricant | 1.0 |
| | | Total layer weight | 200 |
| Total theoretical tablet core weight | | | 775 |
| 12. | Coating | | 38.8 |

| | |
|---|---|
| Total theoretical coated tablet weight | 813.8 |

*Contains 75 mg of Clopidogrel free base

TABLE 1-2
Composition of SCH 53034 Bisulfate-Clopidogrel
Bilayer Tablets; 2.5 mg/75 mg

| | | |
|---|---|---|
| | | Amount/Tablet |
| Ingredient | Function | (mg) |

Clopidogrel Layer

| | | | |
|---|---|---|---|
| 1. | Clopidogrel Premix | Active | 369.5* |
| 2. | Silicified microcrystalline | Compression aid | 125.1 |
| | cellulose | | |
| 3. | Lactose anhydrous | Compression aid | 41.7 |
| 4. | Croscarmellose sodium | Disintegrant | 34.5 |
| 5. | Magnesium stearate | Lubricant | 4.3 |
| | | Total layer weight | 575.0 |

SCH 530348 bisulfate Layer

| 6. | TRA-Bisulfate | Active | 2.5 |
|---|---|---|---|
| 7. | Silicified microcrystalline | Compression aid | 132.4 |
| | cellulose | | |
| 8. | Lactose anhydrous | Compression aid | 44.1 |
| 9. | Croscarmellose sodium | Disintegrant | 12.0 |
| 10. | Hydroxypropyl cellulose | Binder | 8.0 |
| 11. | Magnesium stearate | Lubricant | 1.0 |
| | | Total layer weight | 200 |
| Total theoretical tablet core weight | | | 775 |
| 12. | Coating | | 38.8 |

| | |
|---|---|
| Total theoretical coated tablet weight | 813.8 |

*Contains 75 mg of Clopidogrel free base

TABLE 1-3
Composition of SCH 53034 Bisulfate-Clopidogrel
Bilayer Tablets; 2.5 mg/75 mg

| | | |
|---|---|---|
| | | Amount/Tablet |
| Ingredient | Function | (mg) |

Clopidogrel Layer

| | | | |
|---|---|---|---|
| 1. | Clopidogrel Premix | Active | 375.0* |
| 2. | Silicified microcrystalline | Compression aid | 120.9 |
| | cellulose | | |
| 3. | Lactose anhydrous | Compression aid | 40.3 |
| 4. | Croscarmellose sodium | Disintegrant | 34.5 |
| 5. | Magnesium stearate | Lubricant | 4.3 |
| | | Total layer weight | 575.0 |

SCH 530348 bisulfate Layer

| | | | |
|---|---|---|---|
| 6. | TRA-Bisulfate | Active | 2.5 |
| 7. | Silicified microcrystalline | Compression aid | 130.1 |
| | cellulose | | |
| 8. | Lactose anhydrous | Compression aid | 43.4 |
| 9. | Croscarmellose sodium | Disintegrant | 12.0 |
| 10. | Hydroxypropyl cellulose EFX | Binder | 11.0 |
| 11. | Magnesium stearate | Lubricant | 1.0 |
| | | Total layer weight | 200 |

| | |
|---|---|
| Total theoretical tablet core weight | 775 |

Tablet Coating

| | | | |
|---|---|---|---|
| 12. | OPADRY ® II Yellow | Coating | 23.3 |
| 13. | Purified Water | Solvent | q.s. |

| | |
|---|---|
| Coating theoretical weight | 23.3 mg |
| Theoretical total film-coated bi-layer tablet weight | 798.3 mg |

*Contains 75 mg of Clopidogrel free base

(2): Physical Properties of Clopidogrel Premix
Due to the sticky nature of the clopidogrel free base, the flow and compressibility properties of the resultant premix are poor. Three different excipients were evaluated to enhance the flow and compressibility of the premix as outlined in Table 2. Table 2 demonstrates that silicified microcrystalline cellulose (SMCC) possesses significantly superior flow characteristics when compared to both microcrystalline cellulose (MCC) alone and physical blends of MCC and silicon dioxide (SiO₂). Based on these results, SMCC was chosen over MCC and a mixture of MCC and SiO₂ as the compression aid due to its dual function of improving flow and compressibility of the premix.

TABLE 2
Relative flow index (RFI) comparison
of various materials and blends

| | | | Avicel | Lactose | Silicon | Relative |
|---|---|---|---|---|---|---|
| | Premix | SMCC | PH101 | anhydrous | dioxide | Flow |
| Sample | (%) | (%) | (%) | (%) | (%) | Index |
| Premix | 100 | 0 | 0 | 0 | 0 | 2.2 |
| SMCC | 0 | 100 | 0 | 0 | 0 | 5.2 |
| alone | | | | | | |
| MCC + | 0 | 0 | 98 | 0 | 2 | 3.75 |
| SiO₂ | | | | | | |
| MCC | 0 | 0 | 100 | 0 | 0 | 3.03 |
| alone | | | | | | |
| Premix + | 65 | 35 | 0 | 0 | 0 | 3.26 |
| SMCC | | | | | | |
| Premix + | 65 | 25 | 0 | 10 | 0 | 3.34 |
| SMCC + | | | | | | |
| Lactose | | | | | | |
| Premix + | 65 | 0 | 33 | 0 | 2 | 2.78 |
| MCC + | | | | | | |
| SiO₂ | | | | | | |

(3): Chemical Properties of Clopidogrel Premix

a. Chemical Stability in the Presence of Heat and Humidity
Based on forced degradation data generated at Dr. Reddy's Lab, India it was established that clopidogrel is susceptible to degradation upon exposure to heat, humidity, and oxygen via two major pathways: oxidation (cyclic amide derivative) and inversion (R-enantiomer). Other degradation products of clopidogrel include a dehyro-(oxidative degradation) and a pyridinium degradant. The structures of the aforementioned degradation products are shown below in Scheme 1.
Therefore, excipients with low moisture content (e.g., lactose anhydrous vs. lactose monohydrate) were chosen for formulation development. In addition, dry granulation was determined as the process of choice as opposed to wet granulation to limit the exposure of the clopidogrel premix to high humidity and high temperature during processing.

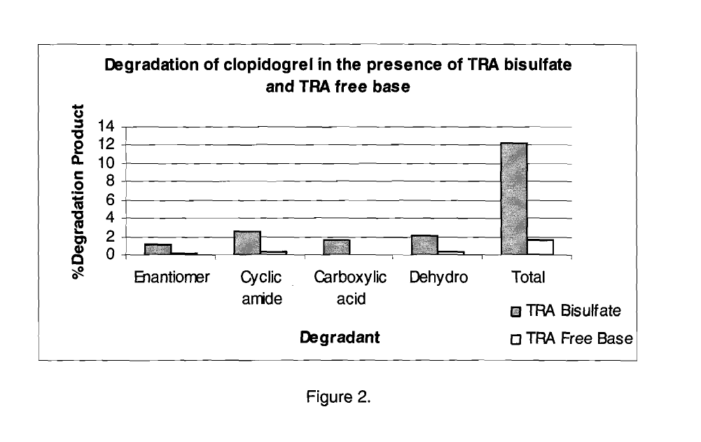

b. Chemical Instability in the Presence of 530348 Bisulfate
Compatibility studies between the clopidogrel premix and 530348 bisulfate were performed to determine the suitability of a monolayer vs. a bilayer tablet. Based on the compatibility results, it was concluded that while 530348 is stable (data not shown) in the presence of clopidogrel, clopidogrel degrades. Clopidogrel is known to degrade under acidic and alkaline conditions. It was hypothesized that 530348 bisulfate presents an acidic microenvironment as a result of which clopidogrel degrades. To test this hypothesis, the above studies were repeated in the presence of 530348 free base. These results (FIG. 2) clearly demonstrate that the extent of clopidogrel degradation is greater in the presence of the bisulfate salt when compared to that of 530348 free base. Therefore, it was concluded that separation of clopidogrel from 530348 bisulfate is necessary for the stability of clopidogrel. Based on this conclusion, prototype tablets with different degrees of separation between the two actives were manufactured and tested for stability.

(4): Prototype Formulations

Three (3) prototype formulations with different degrees of separation between the actives were developed as follows:
1. Single granulation monolayer tablet
2. Separate granulation (530348 bisulfate and clopidogrel granulated separately) monolayer tablet referred to as “separate granulation” tablets
3. Bilayer tablet

The compositions of these three prototypes are shown in Table 3 along with the composition of a prototype tablet containing no 530348 bisulfate. The purpose of developing a tablet formulation with no 530348 bisulfate was to evaluate the stability of clopidogrel in the presence of tablet excipients alone. Thus, the no-530348 bisulfate tablet would allow differentiation in instability due to 530348 bisulfate alone versus instability due to the formulation and process.

TABLE 3
Composition of SCH 530348-clopidogrel (2.5 mg/75 mg) Tablets

| | Amount per tablet (mg) | | | |
|---|---|---|---|---|
| | | Separate | | |
| Ingredient | Monolayer | Granulation | Bilayer | No TRA |
| SCH 530348 | 2.5 | 2.5 | 2.5 | 0 |
| bisulfate | | | | |
| Clopidogrel | 360.6 | 360.6 | 360.6 | 360.6 |
| premix | | | | |
| Silicified | 137 | 211 | 283 | 141 |
| microcrystalline | | | | |
| cellulose | | | | |
| Anhydrous | 46 | 70 | 94 | 47 |
| lactose | | | | |
| Croscarmellose | 23 | 27 | 31 | 23 |
| sodium | | | | |
| Magnesium | 2.8 | 3.4 | 4 | 2.8 |
| Stearate | | | | |
| Silicon dioxide | 3.0 | 0 | 0 | 0 |
| Total Tablet | 575 | 675 | 775 | 575 |
| Weight | | | | |

(5): The stability of the aforementioned prototypes was evaluated after 1 month under International Conference for Harmonization (“ICH”) long-term, ICH intermediate, and ICH accelerated conditions, as well as at 50° C. in 50-mL induction-sealed high density polyethylene (HDPE) bottles. The data are shown in Table 4.

TABLE 4
Prototype Stability Comparison at 4 weeks in 50-mL induction-sealed HDPE Bottles

| | % degradant | | | | | | | |
|---|---|---|---|---|---|---|---|---|
| | Monolayer | | Separate Granulation | | Bilayer | | No TRA | |
| | | Cyclic | | Cyclic | | Cyclic | | Cyclic |
| Condition | Enantiomer | amide | Enantiomer | amide | Enantiomer | amide | Enantiomer | amide |
| Initial | NQ | 0.08 | NQ | 0.08 | ND | 0.09 | NQ | 0.08 |
| 25° C./ | 0.07 | 0.11 | 0.06 | 0.10 | 0.07 | 0.11 | NQ | 0.11 |
| 60% RH | | | | | | | | |
| 30° C./ | 0.16 | 0.12 | 0.10 | 0.11 | 0.10 | 0.12 | 0.09 | 0.13 |
| 65% RH | | | | | | | | |
| 40° C./ | 0.81 | 0.16 | 0.54 | 0.17 | 0.23 | 0.16 | 0.17 | 0.15 |
| 75% RH | | | | | | | | |
| 50° C. | 4.58 | 0.20 | 3.63 | 0.22 | 1.3 | 0.23 | 0.67 | 0.22 |

NQ: Not quantifiable; ND: Not detectable

Based on the 40° C./75% RH and 50° C. results from Table 4, it is concluded that the bilayer tablets are more stable than either one of the monolayer prototypes. Therefore, the bilayer tablets were chosen as one possible avenue for further development.

A desiccant can be added to the primary package containing the tablets.

(6): The stability of a formulation according to the present invention (see Table 5 below) was compared to PLAVIX®, which is commercially available form Bristol-Myers Squibb or Sanofi-Aventis, over a three month period at a temperature of 40° C. and a relative humidity 75%. The stability data are shown in Tables 6 and 7.

TABLE 5
Composition of SCH 530348 Bisulfate-Clopidogrel
Bilayer Tablet; 2.5 mg/75 mg

| | | |
|---|---|---|
| | | Amount/Tablet |
| Ingredient | Function | (mg) |

Clopidogrel Layer

| | | | |
|---|---|---|---|
| 1. | Clopidogrel Premix | Active | 375* |
| 2. | Silicified microcrystalline | Compression aid | 120.89 |
| | cellulose | | |
| 3. | Lactose anhydrous | Compression aid | 40.30 |
| 4. | Croscarmellose sodium | Disintegrant | 34.50 |
| 5. | Magnesium stearate | Lubricant | 4.32 |
| | | Total layer weight | 575.0 |

SCH 530348 bisulfate Layer

| 6. | TRA-Bisulfate | Active | 2.50 |
|---|---|---|---|
| 7. | Silicified microcrystalline | Compression aid | 130.13 |
| | cellulose | | |
| 8. | Lactose anhydrous | Compression aid | 430.38 |
| 9. | Croscarmellose sodium | Disintegrant | 12.00 |
| 10. | Hydroxypropyl cellulose | Binder | 11.00 |
| 11. | Magnesium stearate | Lubricant | 1.00 |
| | | Total layer weight | 200.0 |
| Total theoretical tablet core weight | | | 775.0 |
| 12. | Coating | | 23.25 |

| | |
|---|---|
| Total theoretical coated tablet weight | 798.25 |

*Contains 75 mg of Clopidogrel free base

TABLE 6
Stability Comparison of the Formulation of Table 5

| | | 40° C./75% RH | |
|---|---|---|---|
| Test | Initial | 1 Month | 3 Months |
| Description | Pink | NT | White |
| | Tablet | | Tablet |
| Assay—Clopidogrel free base | 97.7 | 86.9 | 76.4 |
| Clopidogrel Degradation Products | | | |
| Imp B—Cyclic Amide | ND | <RecTh | 0.06 |
| Imp C—Carboxylic Acid | ND | 3.30 | 16.32 |
| Imp D—Dehydro | 0.06 | 0.46 | 0.41 |
| Imp F—Regio Isomer | 0.10 | 0.05 | 0.08 |
| Imp G—Enantiomer | 0.24 | 1.44 | 4.64 |
| Pyridinium Degradation Product | ND | 0.63 | 1.15 |
| Unknown Deg 2 | ND | 0.08 | 0.52 |
| Unknown Deg RRT 0.27 | ND | 0.20 | 0.29 |
| Unknown Deg RRT 0.49 | ND | ND | 0.06 |
| Unknown Deg RRT 0.56 | ND | ND | 0.39 |
| Unknown Deg RRT 0.58 | ND | ND | 0.07 |
| Unknown Deg RRT 0.63 | ND | 0.50 | 0.25 |
| Unknown Deg RRT 0.73 | ND | 2.12 | 2.91 |
| Unknown Deg RRT 0.90 | ND | <RecTh | 0.18 |
| Unknown Deg RRT 0.95 | ND | ND | 0.11 |
| Unknown Deg RRT 1.13 | ND | 0.09 | ND |
| Unknown Deg RRT 1.70 | ND | <RecTh | ND |
| Unknown Deg RRT 1.81 | ND | 0.06 | ND |
| Total Clopidogrel Degradation | 0.40 | 8.93 | 27.44 |
| Products | | | |
| Moisture Content (%) | 1.0 | NT | 5.8 |

Imp = Impurity
ND = Not Detected
NMT = Not more than
RecTh = 0.05%
Repth = 0.08%
NT = Not Tested

TABLE 7
Stability Data of PLAVIX ®

| | 40° C./75% RH | | |
|---|---|---|---|
| Test | Initial | 1 month | 3 months |
| Description | Pink | NT | White |
| | Tablet | | Tablet |
| Assay—Clopidogrel free base | 97.7 | 86.9 | 76.4 |
| Clopidogrel Degradation Product | | | |
| Imp B—Cyclic Amide | ND | <RecTh | 0.06 |
| Imp C—Carboxylic Acid | ND | 3.30 | 16.32 |
| Imp D—Dehydro | 0.06 | 0.46 | 0.41 |
| Imp F—Regio Isomer | 0.10 | 0.05 | 0.08 |
| Imp G—Enantiomer | 0.24 | 1.44 | 4.64 |
| Pyridinium Degradation Product | ND | 0.63 | 1.15 |
| Unknown Deg 2 | ND | 0.08 | 0.52 |
| Unknown Deg RRT 0.27 | ND | 0.20 | 0.29 |
| Unknown Deg RRT 0.49 | ND | ND | 0.06 |
| Unknown Deg RRT 0.56 | ND | ND | 0.39 |
| Unknown Deg RRT 0.58 | ND | ND | 0.07 |
| Unknown Deg RRT 0.63 | ND | 0.50 | 0.25 |
| Unknown Deg RRT 0.73 | ND | 2.12 | 2.91 |
| Unknown Deg RRT 0.90 | ND | <RecTh | 0.18 |
| Unknown Deg RRT 0.95 | ND | ND | 0.11 |
| Unknown Deg RRT 1.13 | ND | 0.09 | ND |
| Unknown Deg RRT 1.70 | ND | <RecTh | ND |
| Unknown Deg RRT 1.81 | ND | 0.06 | ND |
| Total Clopidogrel Degradation | 0.40 | 8.93 | 27.44 |
| Products | | | |
| Moisture Content (%) | 1.0 | NT | 5.8 |

ND = Not Detected
NMT = Not more than
RecTh = 0.05%
RepTh = 0.08%
NT = Not Tested

The data indicate that the formulation presented in Table 5 provides greater stability for clopidogrel relative to PLAVIX®.

Commonly used binders include starch, pre-gelatinized starch, acacia, polyvinylpyrrolidone (“PVP”), hydroxylpropyl cellulose (“HPC”) and hydroxypropyl methylcellulose (“HPMC”) or any combination thereof. In the present invention, the binders preferably comprise between about 2 wt % to about 10 wt % of the solid dosage form.

Disintegrants are used to promote swelling and disintegration of the tablet after exposure to fluids in the oral cavity and/or gastrointestinal tract. Commonly used disintegrants include starch, microcrystalline cellulose, insoluble ion exchange resins, sodium starch glycolate, croscarmelose sodium, alginic acid, sodium alginate, crospovidone and gums, including agar, guar and xanthan. In the present invention, the disintegrant preferably comprises between about 5 wt % and about 10 wt % of the solid dosage form.

Lubricants are used to promote flowability of powders, and to reduce friction between the tablet punch faces and the tablet punches and between the tablet surface and the die wall. Among the most commonly used lubricants are magnesium stearate, calcium stearate, zinc stearate, stearic acid, sodium stearyl fumarate, polyethylene glycol, sodium lauryl sulphate magnesium lauryl sulphate, and sodium benzoate. In the present invention, lubricants preferably comprise 0.25 wt % to 2 wt % of the solid dosage form.

Fillers and compression aids provide bulk and can bind to the active pharmaceutical ingredient, thus reducing the potential for segregation and promoting content uniformity. Commonly used fillers include microcrystalline cellulose, starch, dibasic calcium phosphate dihydrate, lactose, sorbitol, and mannitol. In the present invention, fillers preferably comprise between 5 wt % to 75 wt % of the solid dosage form.

Coatings are used for cosmetic purposes and film-coatings help the swallowability of the dosage form. In the present invention, coatings preferably comprise 1 wt % to 5 wt % of the solid dosage form.

Moisture proof packaging material includes for example aluminum foil blister packs or high density polyethylene (HDPE) bottles.

The formulations of the present invention preferably contain SCH 530348 and/or SCH 530348 bisulfate, described above, in an amount of about 2.5 mg and the clopidogrel, as described above, in an amount of about 75 mg.

### Indications

In some embodiments, the invention is directed to methods of treating acute coronary syndrome or peripheral arterial disease, or of treating a patient in need of secondary prevention by orally administering to a patient in need of such treating the pharmaceutical bilayer tablet.

Thrombin receptor antagonists are disclosed as being useful agents in the treatment of a variety of cardiovascular conditions in U.S. Publication No. 2004/0192753. Thus, among the cardiovascular conditions for which the SCH 530348 bisulfate-clopidogrel bilayer tablet is useful are the following: acute coronary syndrome, peripheral arterial disease, thrombosis, atherosclerosis, restenosis, hypertension, angina pectoris, arrhythmia, heart failure, myocardial infarction, glomerulonephritis, thrombotic stroke, thromboembolic stroke, and cerebral ischemia, deep vein thrombosis, venous thromboembolism, a cardiovascular disease associated with hormone replacement therapy, disseminated intravascular coagulation syndrome, renal ischemia, cerebral stroke, cerebral infarction, migraine, renal vascular homeostasis and erectile dysfunction.

“Secondary prevention” refers to the treatment of patients who have already suffered a significant cardiovascular event, such as a heart attack or stroke, to prevent another future, potentially more serious, perhaps lethal, cardiovascular or cerebrovascular event.

Thrombin receptor antagonists can be useful in the prevention of cardiovascular events associated with cardiopulmonary bypass surgery, as described in U.S. Publication No. 2007/0202140. SCH 530348 bisulfate-clopidogrel bilayer bilayer tablet may be a particularly effective agent in such use. Thus, the present invention is directed to a method of preventing a condition associated with coronary arterial bypass graft surgery comprising administering a SCH 530348 bisulfate-clopidogrel bilayer tablet to a subject of said surgery. In some embodiments, the condition is selected from the group consisting of: bleeding; thrombotic vascular events such as thrombosis, restenosis; vein graft failure; artery graft failure; atherosclerosis, angina pectoris; myocardial ischemia; acute coronary syndrome myocardial infarction; heart failure; arrhythmia; hypertension; transient ischemic attack; cerebral function impairment; thromboembolic stroke; cerebral ischemia; cerebral infarction; thrombophlebitis; deep vein thrombosis; and, peripheral vascular disease.

The use of thombin receptor antagonists to control the risk of bleeding events in patients undergoing non-emergent percutaneous coronary intervention is disclosed in U.S. Publication No. 2008/0234236. Thus, within the scope of the present invention are methods of preventing a major cardiac event (e.g., myocardial infarction, urgent revascularization, or ischemia requiring hospitalization) in a patient who has undergone percutaneous coronary intervention comprising administering a pharmaceutical formulation comprising SCH530348 and/or SCH 530348 bisulfate, chlopidogrel and silicified microcrystalline cellulose, including, for example, SCH 530348 bisulfate-clopidogrel bilayer tablet, to the patient. Also within the inventive scope are methods of inhibiting TRAP-induced platelet aggregation, which may or may not be associated with PCI.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A pharmaceutical formulation which comprises
a) a compound of the formula: 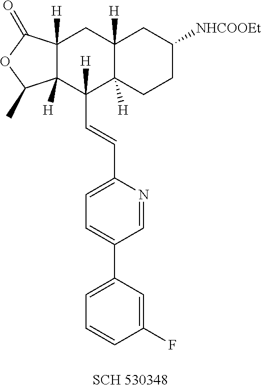
or the bisulfate salt thereof;
b) clopidogrel; and
c) silicified microcrystalline cellulose.

2. The pharmaceutical formulation of claim 1 which is a tablet.

3. The pharmaceutically formulation of claim 2 the compound is SCH 530348.

4. A pharmaceutical tablet which comprises: 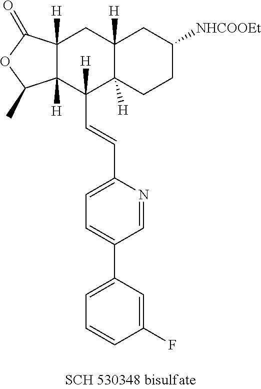
and clopidogrel and silicified microcrystalline cellulose.

5. The tablet of claim 4, wherein said tablet is a bilayer tablet.

6. The bilayer tablet of claim 5 comprising a first layer containing SCH 530348 bisulfate and a second layer containing clopidogrel.

7. The bilayer tablet of claim 6, further comprising one or more excipients.

8. The bilayer tablet of claim 7, wherein the excipients of said first layer is selected from the group consisting of anhydrous lactose, silicified microcrystalline cellulose, sodium croscarmellose, hydroxypropyl cellulose and magnesium stearate; and the excipients of said second layer is selected from the group consisting of butylated hydroxyl anisole, mannitol, anhydrous lactose, micorocyrstalline cellulose, aerosol, silicified microcrystalline cellulose, sodium croscarmellose, and magnesium stearate.

9. The bilayer tablet of claim 6 wherein the amount of SCH 530348 bisulfate in said first layer is about 2.5 mg and the amount of clopidogrel in said second layer is about 75 mg.

10. A method of treating a cardiovascular condition comprising administering to a mammal in need of such treatment an effective amount of the pharmaceutical formulation of claim 1.

11. A method of treating a cardiovascular condition comprising administering to a mammal in need of such treatment an effective amount of the tablet of claim 4.

12. The method according to claim 11 wherein said cardiovascular condition is selected from the group consisting of acute coronary syndrome, peripheral arterial disease, thrombosis, atherosclerosis, restenosis, hypertension, angina pectoris, arrhythmia, heart failure, myocardial infarction, glomerulonephritis, thrombotic stroke, thromboembolic stroke, and cerebral ischemia.

13. A method of preventing a condition associated with coronary arterial bypass graft surgery comprising administering to a subject of said surgery a tablet according to any of claim 4.

14. A method of preventing a major cardiac event in a patient who has undergone percutaneous coronary intervention and is in need of such prevention comprising administering an effective amount of a tablet claim 4 to the patient.

15. A bilayer pharmaceutical tablet of claim 4 packaged in a moisture proof packaging material.
